(19) United States
(12) Patent Application Publication
(10) Pub. No.: US 2013/0239708 A1
(43) Pub. Date: Sep. 19, 2013

(54) VALVE-TO-BE-SERVICED SELECTING DEVICE AND METHOD

(71) Applicant: AZBIL CORPORATION, Tokyo (JP)

(72) Inventors: Masato TANAKA, Tokyo (JP); Kenichi KIKUCHI, Tokyo (JP); Isao YAMADA, Tokyo (JP)

(73) Assignee: AZBIL CORPORATION, Tokyo (JP)

(21) Appl. No.: 13/794,987

(22) Filed: Mar. 12, 2013

(30) Foreign Application Priority Data
Mar. 14, 2012 (JP) 2012-056757

(51) Int. Cl. G01N 33/00

(52) U.S. Cl. CPC G01N 33/00
USPC 73/865.8

(57) ABSTRACT

A valve-to-be-serviced selecting device is provided with: a simple identification executing portion identifying a service-candidate valve by a method that is relatively non-labor-intensive (a relatively low-cost method), a fine identification executing portion identifying a service-candidate valve by a method that is relatively labor-intensive (a relatively high-cost method), and a valve-to-be-serviced determination processing portion. The valve-to-be-serviced determination processing portion performs the simple identification by the simple identification executing portion for valves 1-1 through 1-n (N valves), and then performs fine identification, through the fine identification executing portion service-candidate valves identified by the simple identification (M valves), to determine those valves to be subjected to servicing from among the valves 1-1 through 1-n.

### CROSS-REFERENCE TO RELATED APPLICATION

This application claims priority to Japanese Patent Application No. 2012-056757, filed Mar. 14, 2012. The entirety of this application is incorporated herein by reference.

### FIELD OF TECHNOLOGY

The present invention relates to a valve-to-be-serviced selecting device and method, to select a valve to be subjected to servicing from a large number of valves.

### BACKGROUND

Conventionally, large numbers of valves are used in petrochemical, chemical, and other plants, and when performing periodic servicing, a large number of valves are candidates for servicing. When determining (selecting) a valve that is actually going to be subjected to servicing (e.g., an overhaul inspection) from the large number of valves, the party who issues the service order and the party who receives the service order have conferred based on reference data such as time of use. There have been proposals also for equipment information management systems for collecting this reference information and outputting it in the form of a report. (See, for example, Japanese Unexamined Patent Application Publication 2011-215917.)

The large number of valves used in a plant is a mixture of valves for which an overhaul inspection is imperative, and, conversely, valves for which servicing work is essentially unnecessary. Because of this, selecting, from a large number of valves, the valves that are to be subjected to servicing involves an issue of a trade-off between cost of servicing (labor, replacement materials costs, and the like), and effectiveness of servicing (safety, performance, and the like).

However, in a procedure based on such discussion, the handling of cost versus effectiveness (cost-performance) when it comes to servicing is haphazard, which may produce cases that are handled remarkably poorly. For example, there are those cases wherein a great deal of time is spent in discussion in order to determine the valve that is to be serviced, time usage that may be considered to be unnecessary.

The present invention solves such a problem, and the object thereof is to provide a valve-to-be-serviced selecting device and selecting method for providing a standardized procedure for selecting, from a large number of valves, a valve to be subjected to servicing, taking cost-performance into account.

### SUMMARY

In order to achieve the object set forth above, the example of the present invention is a valve-to-be-serviced selecting device including simple identification executing means for identifying a service-candidate valve through a method that is relatively non-labor-intensive; fine identification executing means for identifying a service-candidate valve through a method that is relatively labor-intensive; valve-to-be-serviced determining means for determining a valve to be serviced from among a large number of valves by performing identification of a service-candidate valve through the simple identification executing means for a large number of valves and then performing service-candidate valve identification by the fine identification executing means for the service-candidate valves identified by the simple identification executing means.

There is a broad range of information, from (1) simple and clear information such as time of use (information obtained through methods that are relatively non-labor-intensive (relatively low-cost methods)) through (2) sophisticated information obtained through online data analysis or through obtaining data through testing activities (information obtained through relatively labor-intensive methods (relatively high-cost methods)) that can be used as the basis for identifying whether or not to perform servicing (in particular, an overhaul inspection) on a valve. Moreover, there is a large variability between valves, such as there being valves that can be excluded from servicing through identification based solely on the simple and clear information. Moreover, when operations were performed on all valves without specifically establishing identification procedures, there are cases wherein the high-cost identification is applied to the valves wherein low-cost identification would have been possible, which has an impact on the cost-performance problem. Given this, in order to make improvements regarding the cost-performance problem when it comes to servicing, it can be effective to perform a procedure to determine which valves are ultimately to be serviced by categorizing the methods into low-cost identification methods and high-cost identification methods, and sequentially applying the methods starting with the low-cost identification methods, to reduce the scope over which the high-cost identification methods are applied.

Based on this technical concept, in the examples of the present invention, simple identification executing means for identifying service-candidate valves by relatively non-labor-intensive methods (relatively low-cost methods) and fine identification executing means for identifying service-candidate valves by relatively labor-intensive methods (relatively high-cost methods) are used to narrow in on the service-candidate valves in a stepwise manner to determine the valves that are ultimately to be subjected to servicing. In this case, identification (simple identification) of service-candidate valves is first performed using the simple identification executing means on a large number of valves. Following this, identification (fine identification) of service-candidate valves is performed by the fine identification executing means for the service-candidate valves identified by the simple identification. Doing this determines the valves that will ultimately be subjected to servicing, through reducing the scope to which the high-cost identification methods are applied through sequentially applying the identification methods starting with the low-cost methods. Doing so improves the cost-performance for the party issuing the service order, and reduces ambiguity regarding liability for maintenance through performing service for the party that has received the service order, thereby making it possible to reduce business risk.

In the example of the present invention, the simple identification executing means and/or fine identification executing means may perform identifications in multiple steps. For example, when performing a simple identification, service-candidate valves may be identified in a first stage based on the number of days that have elapsed since the date on which the most recent service was performed, and then, in a second stage, service-candidate valves may be identified based on the cumulative valve sliding distance since the most recent replacement of the packing. In the case of the fine identification, the service-candidate valves may be identified in a first stage based on the frequency with which stick-slip occurs, and, in a second stage, service-candidate valves may be identified based on step-response characteristics.

Given the examples of the present invention, after identifying service-candidate valves by performing simple identification, fine identification is then performed for the service-candidate valves that have been identified, to determine the valves to be subject to servicing, and thus a standardized procedure for selecting valves to be subject to servicing, which takes cost-performance into account, for a large number of valves is provided, thus improving the cost-performance for the party that issues the service order, and reducing the ambiguity in liability for maintenance due to the execution of service for the party receiving the service order, thus making it possible to reduce business risk.

### BRIEF DESCRIPTIONS OF THE DRAWINGS

FIG. 1 is a configuration diagram of a system that uses an example of a device for selecting a valve to be subjected to servicing, according to the present invention.

FIG. 2 is a flowchart for explaining the processing operations for the device for selecting the valve to be subjected to servicing in an example.

FIG. 3 is a configuration diagram of a system that uses another example of a device for selecting a valve to be subjected to servicing, according to the present invention.

FIG. 4 is a flowchart for explaining the processing operations for the device for selecting the valve to be subjected to servicing in the other example.

FIG. 5 is a diagram illustrating an example of step-response characteristics.

FIG. 6 is a diagram for explaining the details of a control indicator in the step-response characteristics.

### DETAILED DESCRIPTION

Examples according to the present invention are explained below in detail, based on the drawings.

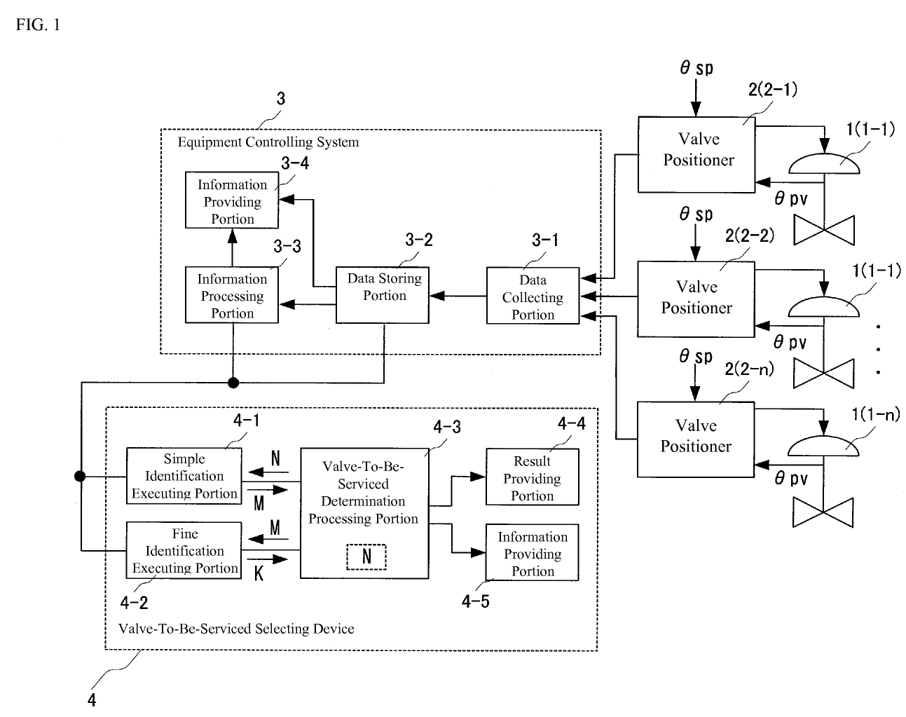

FIG. 1 is a configuration diagram of a system that uses an example of a device for selecting a valve to be subjected to servicing, according to the present invention. This example explains an extremely basic configuration and processing procedure.

In FIG. 1, 1-1 through 1-n are a large number of valves used in a plant, 2-1 through 2-n are positioners (valve positioners) installed in the valves 1-1 through 1-n, 3 is a device management system, and 4 is a valve-to-be-serviced selecting device, according to the example of the present invention.

In this system, a positioner 2 (2-1 through 2-n) is input with a setting opening θsp from a higher-level device (not shown), and controls the opening of the valve 1 (1-1 through 1-n) so that θsp goes to θpv while monitoring the actual opening θpv, which is fed back from the valve 1 (1-1 through 1-n). Moreover, the positioner 2 (2-1 through 2-n) sends, at specific intervals, data pertaining to the control status of the valve 1 (1-1 through 1-n) to the device management system 3.

The device management system 3 is provided with a data collecting portion 3-1, a data storing portion 3-2, a data processing portion 3-3, and a data presenting portion 3-4. The data collecting portion 3-1 collects data from the positioners 2-1 through 2-n, storing it in the data storing portion 3-2. The data processing portion 3-3 performs data processing using the data that has been stored in the data storing portion 3-2, to produce secondary data. The data presenting portion 3-4 presents the data that has been stored in the data storing portion 3-2 and the data generated by the data processing portion 3-3.

The valve-to-be-serviced selecting device 4 is achieved through hardware having a processor and a storage device and a program for achieving a variety of functions in cooperation with this hardware, and is provided with a simple identification executing portion 4-1, a fine identification executing portion 4-2, a valve-to-be-serviced determination processing portion 4-3, a result presenting portion 4-4, and an information presenting portion 4-5.

The valve-to-be-serviced selecting device 4 and the device management system 3 are connected together, and the data that is stored in the data storing portion 3-2 of the device management system 3 and the data generated by the data processing portion 3-3 are used as appropriate by the simple identification executing portion 4-1 and the fine identification executing portion 4-2 within the valve-to-be-serviced selecting device 4.

Note that the valve-to-be-serviced selecting device 4 according to the example of the present invention may be installed on a personal computer (PC) that is the same PC on which, for example, the device management system 3 is installed, and may share data and information therewith, or may be installed on a separate PC and share data and information through a communication function.

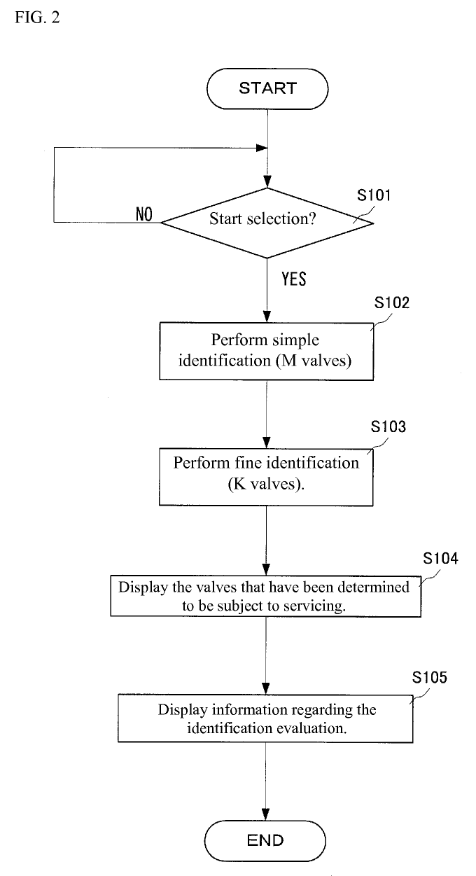

The flowchart shown in FIG. 2 is referenced below to explain the processing operations that are unique to the present example while focusing on the functions of the various portions in the valve-to-be-serviced selecting device 4. Note that the processing operations are performed primarily by the valve-to-be-serviced determination processing portion 4-3. Note that valves 1-1 through 1-n that are subject to selection are registered in advance in the valve-to-be-serviced determination processing portion 4-3. That is, if there are N individual valves 1-1 through 1-n, then N valves are registered as the initial service-candidate valves.

When an instruction for beginning a selection is inputted by an operator (Step S 101: YES), the valve-to-be-serviced determination processing portion 4-3 first sends a command to the simple identification executing portion 4-1, to cause the execution of identification of service-candidate valves by the simple identification executing portion 4-1 (Step S 102).

Identification of Service-Candidate Valves by the Simple Identification Executing Portion (Simple Identification)

The simple identification executing portion 4-1 receives the command from the valve-to-be-serviced determination processing portion 4-3 to identify, as service-candidate valves, those valves that are not excluded from being subjected to servicing (or stated conversely, to identify those valves that can be excluded from being subjected to servicing), of all of the N valves that are registered in the valve-to-be-serviced determination processing portion 4-3. Doing so narrows down the service-candidate valves from N valves to M valves (where N≧M).

The simple identification executing portion 4-1 identifies the service-candidate valves using a method that is relatively non-labor-intensive (i.e., a relatively low-cost method) (that is, performs simple identification). For example, valves for which the number of days that have elapsed since the date on which the most recent service was performed exceeds a reference day count, that is established in advance, are identified as valves that cannot be excluded from being subject to servicing. However, because this is the simple identification stage, preferably the reference day count is established on the low side, so as to provide a safety margin.

The valve-to-be-serviced determination processing portion 4-3 next receives the identification result of the simple identification executing portion 4-1 and sends a command to the fine identification executing portion 4-2 to cause execution of service-candidate valve identification by the fine identification executing portion 4-2 (Step S 103).

Identification of Service-Candidate Valves by the Fine Identification Executing Portion (Fine Identification)

The fine identification executing portion 4-2 receives the command from the valve-to-be-serviced determination processing portion 4-3 to identify, as service-candidate valves, those valves that are not excluded from being subjected to servicing (or stated conversely, to identify those valves that can be excluded from being subjected to servicing), of the valves identified by the simple identification executing portion 4-1 as service-candidate valves, that is, of the M service-candidate valves narrowed in on by the simple identification. Doing so narrows down the service-candidate valves from M valves to K valves (where M≧K).

The fine identification executing portion 4-2 identifies the service-candidate valves using a method that is relatively labor-intensive (i.e., a relatively high-cost method) (that is, performs fine identification). For example, those valves wherein the cumulative valve sliding distance since the most recent packing replacement exceeds a reference distance that has been set in advance are identified as valves that cannot be excluded from being subject to servicing. If the data or information that can be obtained from the device management system 3 is, for example, only a record of the date on which the valve packing was replaced and a history of the valve position command values from the positioner 2, then, for the cumulative valve sliding distance, a process would be required for calculating the sliding distances based thereon. That is, when compared to just the number of days elapsed from the date on which the most recent servicing was performed, this is a method that is labor intensive, a high-cost identifying method. The cost is particularly high, as labor for the operator, when it is the responsibility of the operator to check the results of the calculation.

The valve-to-be-serviced determination processing portion 4-3 receives the identification result from the fine identification executing portion 4-2 and defines, as valves that are subject to servicing, the K valves that have been identified as service-candidate valves, and presents them on the result presenting portion 4-4 to enable these K valves to be recognized as valves to be subject to servicing (Step S104). For example, tag names of the valves are displayed.

Moreover, the valve-to-be-serviced determination processing portion 4-3 presents, on the information presenting portion 4-5, the information used by the simple identification executing portion 4-1 and the fine identification executing portion 4-2 (the number of days elapsed since the date on which the most recent servicing was performed and the cumulative valve sliding distance since the most recent packing replacement) as information relating to the identification evaluation corresponding to the individual valves (Step S105). For example, the information identified as not being excluded from being subject to servicing may be displayed in red text, and the information identified as being able to be excluded from being subject to servicing may be displayed in green text.

The processes set forth above make it possible to exclude N-M valves from being subject to execution of the fine identification, that is, they reduce the number of high-cost identifications performed, which, for the party that issues the service orders, improves the cost-performance, and, for the party that receives the service orders, reduces the ambiguity in the liability for maintenance through performing servicing, making it possible to reduce business risk. Focusing on a low-cost identification method and a high-cost identification method by merely discriminating in stages, and specifying that the low-cost identification method is to be performed first, produces this effect.

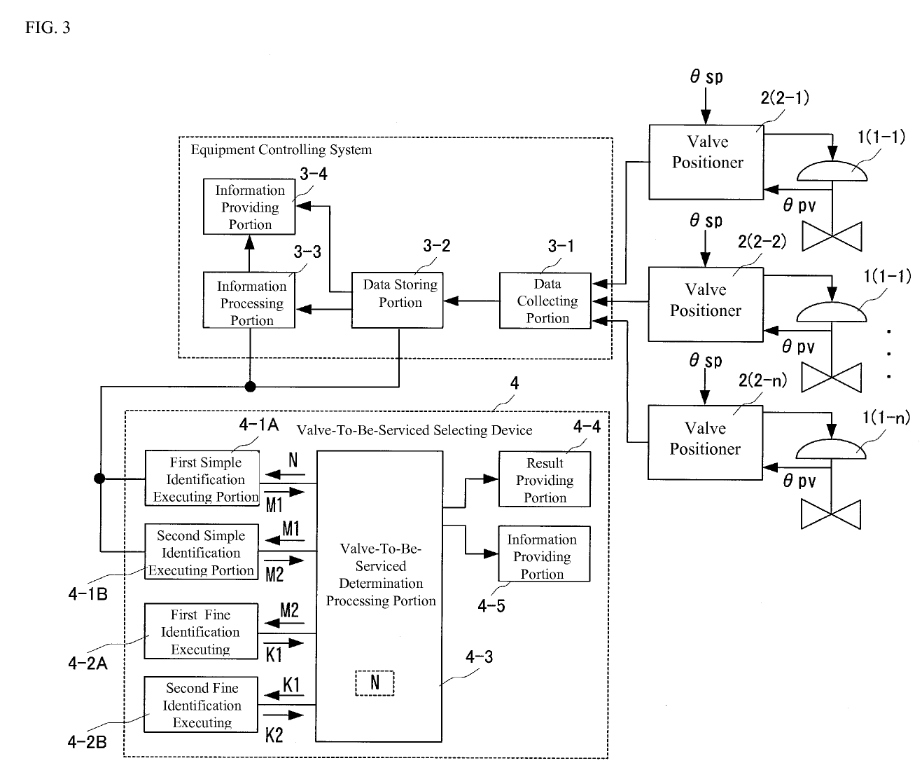

FIG. 3 is a configuration diagram of a system that uses another example of a device for selecting a valve to be subjected to servicing, according to the present invention. The other example illustrates an “identification method that is recommended in practice,” not described above. Note that this is to illustrate that the identification of service-candidate valves is not limited to two stages (that is, neither the simple identification nor the fine identification is limited to a single stage). Note, however, that the identification method described in the present example is no more than an example. Note that, in FIG. 3, codes identical to those in FIG. 1 indicate structural elements that are identical or equivalent to structural elements explained in reference to FIG. 1, and explanations thereof are omitted.

In the other example, the valve-to-be-serviced selecting device 4 is provided with a first simple identification executing portion 4-1A, a second simple identification executing portion 4-1B, a first fine identification executing portion 4-2A, a second fine identification executing portion 4-2B, a valve-to-be-serviced determination processing portion 4-3, a result presenting portion 4-4, and an information presenting portion 4-5.

In the valve-to-be-serviced selecting device 4, the first simple identification executing portion 4-1A and the second simple identification executing portion 4-1B identify the service-candidate valves using methods that are relatively non-labor-intensive (relatively low-cost methods) (that is, performs simple identification). Note that, in terms of costs, the first simple identification executing portion 4-1A performs identification of service-candidate valves at a lower cost than that of the second simple identification executing portion 4-1B. That is, the first simple identification executing portion 4-1A performs a simple identification (hereinafter termed the “first simple identification”) that is in first place in low-cost order, and the second simple identification executing portion 4-1B performs a simple identification (hereinafter termed the “second simple identification”) that is in second place in low-cost order.

Moreover, in the valve-to-be-serviced selecting device 4, the first fine identification executing portion 4-2A and the second fine identification executing portion 4-2B identify the service-candidate valves using methods that are relatively labor-intensive (relatively high-cost methods) (that is, performs fine identification). Note that, in terms of costs, the first fine identification executing portion 4-2A performs identification of service-candidate valves at a lower cost than that of the second fine identification executing portion 4-2B. That is, the first fine identification executing portion 4-2A performs a fine identification (hereinafter termed the “first fine identification”) that is in third place in low-cost order, and the second fine identification executing portion 4-2B performs a fine identification (hereinafter termed the “second fine identification”) that is in fourth place in low-cost order.

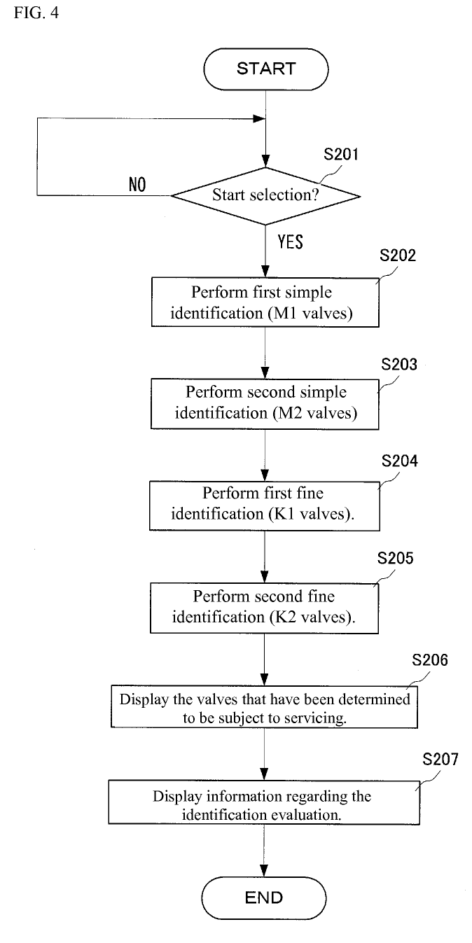

The flowchart shown in FIG. 4 is referenced below to explain the processing operations that are unique to the present example while focusing on the functions of the various portions in the valve-to-be-serviced selecting device 4. Note that the processing operations are performed primarily by the valve-to-be-serviced determination processing portion 4-3, in the same manner as in the above example. Note that valves 1-1 through 1-n that are subject to selection are registered in advance in the valve-to-be-serviced determination processing portion 4-3, in the same manner as in the above example. That is, N valves are registered as the initial service-candidate valves.

When an instruction for beginning a selection is inputted by an operator (Step S201: YES), the valve-to-be-serviced determination processing portion 4-3 first sends a command to the first simple identification executing portion 4-1A, to cause the execution of identification of service-candidate valves by the first simple identification executing portion 4-1A (Step S202).

Identification of Service-Candidate Valves by the First Simple Identification Executing Portion (First Simple Identification)

The first simple identification executing portion 4-1A receives the command from the valve-to-be-serviced determination processing portion 4-3 to identify, as service-candidate valves, those valves that are not excluded from being subjected to servicing, of all of the N valves that are registered in the valve-to-be-serviced determination processing portion 4-3. Doing so narrows down the service-candidate valves from N valves to M1 valves (where N≧M1).

For example, the first simple identification executing portion 4-1A, as the first simple identification (in first place in low-cost order) identifies, as valves that cannot be excluded from being subject to servicing, valves for which the number of days that have elapsed since the date on which the most recent service was performed exceeds a reference day count that is established in advance. However, because this is the simple identification stage, preferably the reference day count is established on the low side, so as to provide a safety margin.

The valve-to-be-serviced determination processing portion 4-3 next receives the identification result of the first simple identification executing portion 4-1A and sends a command to the second simple identification executing portion 4-1B to cause execution of service-candidate valve identification by the second simple identification executing portion 4-1B (Step S203).

Identification of Service-Candidate Valves by the Second Simple Identification Executing Portion (Second Simple Identification)

The second simple identification executing portion 4-1B receives the command from the valve-to-be-serviced determination processing portion 4-3 to identify, as service-candidate valves, those valves that are not excluded from being subjected to servicing, of the valves identified by the first simple identification executing portion 4-1A as service-candidate valves, that is, of the M1 service-candidate valves narrowed in on by the first simple identification. Doing so narrows down the service-candidate valves from M1 valves to M2 valves (where M1≧M2).

For example, the second simple identification executing portion 4-1B, as the second simple identification (in second place in low-cost order) identifies, as valves that cannot be excluded from being subject to servicing, valves for which the cumulative valve sliding distance since the most recent packing replacement exceeds a reference distance that is established in advance. Note that the second simple identification is identical to the fine identification described in the first form of embodiment. That is, the second simple identification is relatively more costly than the first simple identification.

The valve-to-be-serviced determination processing portion 4-3 next receives the identification result of the second simple identification executing portion 4-1B and sends a command to the first fine identification executing portion 4-2A to cause execution of service-candidate valve identification by the first fine identification executing portion 4-2A (Step S204).

Identification of Service-Candidate Valves by the First Fine Identification Executing Portion (First Fine Identification)

The first fine identification executing portion 4-2A receives the command from the valve-to-be-serviced determination processing portion 4-3 to identify, as service-candidate valves, those valves that are not excluded from being subjected to servicing, of the valves identified by the second simple identification executing portion 4-1B as service-candidate valves, that is, of the M2 service-candidate valves narrowed in on by the second simple identification. Doing so narrows down the service-candidate valves from M2 valves to K1 valves (where M2≧K1).

For example, the first fine identification executing portion 4-2A, as the first fine identification (in third place in low-cost order) identifies, as valves that cannot be excluded from being subject to servicing, valves for which the number of stick-slit occurrences per reference sliding distance (that is, the occurrence frequency) exceeds a reference frequency that is established in advance. Because in the first fine identification the number of occurrences of stick-slip per reference sliding distance is calculated after calculating the sliding distance, this is an identification method that has a higher cost than the sliding distance. Note that the detection of stick-slip is disclosed in Japanese Patent 3254624, so detailed explanations thereof are omitted here.

The valve-to-be-serviced determination processing portion 4-3 next receives the identification result of the first fine identification executing portion 4-2A and sends a command to the second fine identification executing portion 4-2B to cause execution of service-candidate valve identification by the second fine identification executing portion 4-2B (Step S205).

Identification of Service-Candidate Valves by the Second Fine Identification Executing Portion (Second Fine Identification)

The second fine identification executing portion 4-2B receives the command from the valve-to-be-serviced determination processing portion 4-3 to identify, as service-candidate valves, those valves that are not excluded from being subjected to servicing, of the valves identified by the first fine identification executing portion 4-2A as service-candidate valves, that is, of the K1 service-candidate valves narrowed in on by the first fine identification. Doing so narrows down the service-candidate valves from K1 valves to K2 valves (where K1≧K2).

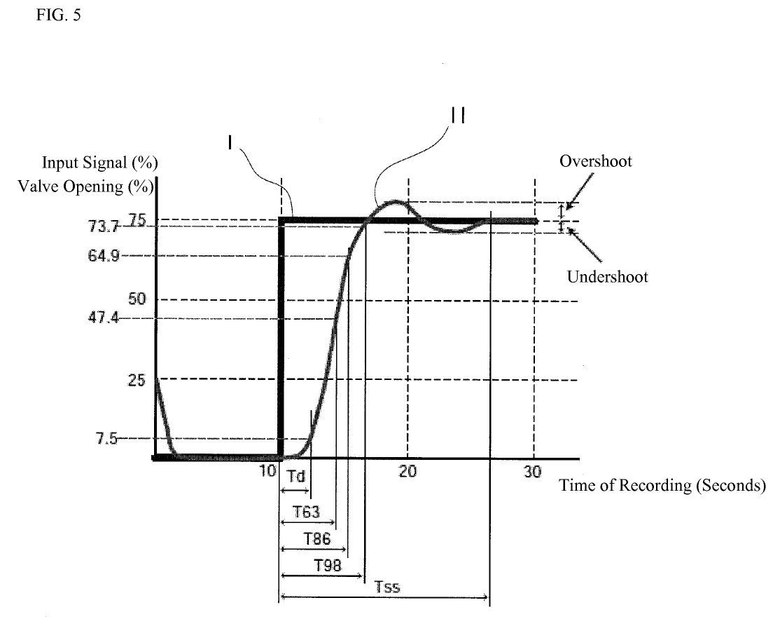

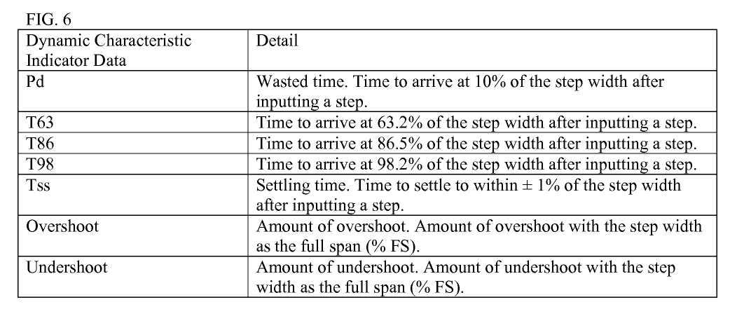

The second fine identification executing portion 4-2B identifies the service-candidate valves using step response characteristics, for example, as the second fine identification (in fourth place in low-cost order). An example of step response characteristics is illustrated in FIG. 5. In FIG. 5, I is a step-shaped input waveform, and II is the waveform of the response to the step-shaped input waveform (the step-response characteristics). The step-response characteristics include a plurality of control indicators such as the response times Td, T63, T86, and T98, the settling time Tss, overshoot, undershoot, and the like (referencing FIG. 6), where the step-response operations for obtaining the control indicators are performed automatically off-line, making this the highest-cost identification method. In the second fine identification, at least one of these control indicators is used to identify, as valves that cannot be excluded from being subject to servicing, those wherein a control indicator is outside of the normal range, established in advance. Note that the function for performing the step-response operations automatically off-line is commonly achieved by the valve, positioner, and device management system, and is a well-known technology.

The valve-to-be-serviced determination processing portion 4-3 receives the identification result from the second fine identification executing portion 4-2B and defines, as valves that are subject to servicing, the K2 valves that have been identified as service-candidate valves, and presents them on the result presenting portion 4-4 to enable these K2 valves to be recognized as valves to be subject to servicing (Step S206). For example, tag names of the valves are displayed.

Moreover, the valve-to-be-serviced determination processing portion 4-3 presents, on the information presenting portion 4-5, the information used by the first simple identification executing portion 4-1A, the second simple identification executing portion 4-1B, the first fine identification executing portion 4-2A, and the second fine identification executing portion 4-2B (the number of days elapsed since the date on which the most recent servicing was performed, the cumulative valve sliding distance since the most recent packing replacement, the number of occurrences of stick-slip per reference sliding distance, and the control indicator for the step-response characteristics) as information relating to the identification evaluation corresponding to the individual valves (Step S207). For example, the information identified as not being excluded from being subject to servicing may be displayed in red text, and the information identified as being able to be excluded from being subject to servicing may be displayed in green text.

The processes set forth above make it possible to exclude N-M2 valves from being subject to execution of the first fine identification, and possible to exclude N-K1 valves from being subject to execution of the second fine identification, that is, they reduce the number of high-cost identifications performed, which, for the party that issues the service orders, improves the cost-performance, and, for the party that receives the service orders, reduces the ambiguity in the liability for maintenance through performing servicing, making it possible to reduce business risk. Focusing on a low-cost identification method and a high-cost identification method by merely discriminating in stages, and specifying that the low-cost identification method is to be performed first, produces this effect.

While examples of the present invention have been explained above, the present invention is not limited to the examples set forth above. The structures and details in the present invention may be varied in a variety of ways, as can be understood by one skilled in the art, within the scope of technology in the present invention.

We claim:

1. A valve-to-be-serviced selecting device comprising:
a simple identification executing device identifying a service-candidate valve through a method that is relatively non-labor-intensive;
a fine identification executing device identifying a service-candidate valve through a method that is relatively labor-intensive;
a valve-to-be-serviced determining device determining a valve to be serviced from among a large number of valves by performing identification of a service-candidate valve through the simple identification executing device on a large number of valves and then performing service-candidate valve identification by the fine identification executing device on the service-candidate valves identified by the simple identification executing means.

2. The valve-to-be-serviced selecting device as set forth in claim 1, further comprising:
an information presenting device presenting information regarding the identification evaluation of a service-candidate valve in the simple identification executing device and the fine identification executing device.

3. The valve-to-be-serviced selecting device as set forth in claim 1, wherein:
the simple identification executing device identifies a service-candidate valve based on the number of days elapsed since the date on which the most recent service was performed.

4. The valve-to-be-serviced selecting device as set forth in claim 1, wherein:
either the simple identification executing device or the fine identification executing device identifies a service-candidate valve based on a cumulative valve sliding distance since the most recent packing replacement.

5. The valve-to-be-serviced selecting device as set forth in claim 1, wherein:
either the simple identification executing device or the fine identification executing device identifies a service-candidate valve based on a frequency with which stick-slip occurs.

6. The valve-to-be-serviced selecting device as set forth in claim 1, wherein:
the fine identification executing device identifies a service-candidate valve based on step-response characteristics.

7. A valve-to-be-serviced selecting method comprising:
a simple identification executing step identifying a service-candidate valve through a method that is relatively non-labor-intensive;
a fine identification executing step identifying a service-candidate valve through a method that is relatively labor-intensive;
a valve-to-be-serviced determining step determining a valve to be serviced from among a large number of valves by performing identification of a service-candidate valve through the simple identification executing step for a large number of valves and then performing service-candidate valve identification by the fine identification executing step for the service-candidate valves identified by the simple identification executing step.

8. The valve-to-be-serviced selecting method as set forth in claim 7, further comprising:
an information presenting step presenting information regarding the identification evaluation of a service-candidate valve in the simple identification executing step and the fine identification executing step.

9. The valve-to-be-serviced selecting method as set forth in claim 7, wherein:
the simple identification executing step identifies a service-candidate valve based on the number of days elapsed since the date on which the most recent service was performed.

10. The valve-to-be-serviced selecting method as set forth in claim 7, wherein:
either the simple identification executing step or the fine identification executing step identifies a service-candidate valve based on a cumulative valve sliding distance since the most recent packing replacement.

11. The valve-to-be-serviced selecting method as set forth in claim 7, wherein:
either the simple identification executing step or the fine identification executing step identifies a service-candidate valve based on a frequency with which stick-slip occurs.

12. The valve-to-be-serviced selecting method as set forth in claim 7, wherein:
the fine identification executing step identifies a service-candidate valve based on step-response characteristics.
